(19) United States
(12) Patent Application Publication
(10) Pub. No.: US 2023/0329308 A1
(43) Pub. Date: Oct. 19, 2023

(54) COMPOSITIONS COMPRISING A MINERAL SALT FOR ORAL USE

(71) Applicant: DIFASS INTERNATIONAL S.P.A., Coriano (RN) (IT)

(72) Inventors: Alida Agostini, Coriano (Rn) (IT); Davide Giorgini, Savignano sul Rubicone (FC) (IT)

(73) Assignee: DIFASS INTERNATIONAL S.P.A., Coriano (RN) (IT)

(21) Appl. No.: 18/245,615

(22) PCT Filed: Sep. 16, 2021
(86) PCT No.: PCT/IB2021/058450

§ 371 Date: Mar. 16, 2023

(30) Foreign Application Priority Data
Sep. 18, 2020 (IT) 102020000022003

(51) Int. Cl. A23L 33/29; A23L 29/269; A23L 33/28

(52) U.S. Cl. CPC A23L 33/29; A23L 29/273; A23L 33/28

(57) ABSTRACT

Disclosed is a composition for oral use in the form of a granulate or micro-granule comprising a mineral salt combined with soluble fibre, said granulate or micro-granule being coated with sodium alginate and shellac.

The invention relates to a composition for oral use in the form of a granulate or micro-granule comprising a mineral salt combined with soluble fibre, said granulate or micro-granule being coated with shellac and sodium alginate.

### PRIOR ART

Mineral salts are inorganic substances normally supplied by foodstuffs. Unlike carbohydrates, fats and proteins, they do not supply energy, but are nevertheless essential to health. Said mineral salts are elements physiologically present in varying amounts, depending on the concentrations necessary for the development and growth of the human body. A sufficient intake enables the body to perform its functions correctly. An insufficient intake caused, for example, by an incorrect or unbalanced diet or increased requirements, can lead to serious health consequences.

The proportion of mineral salts present in a certain food does not always correspond to the bioavailable proportion, or to the proportion actually metabolised and used by the body. For example, some mineral salts can have bioavailability problems when they bind to phytates, substances commonly present in foods like pulses and cereals, which prevent their absorption. Thus, certain types of diet, such as a vegetarian or vegan diet, can make it difficult to achieve a correct intake of mineral salts, and with time can lead to deficiencies that endanger the health.

The use of products containing mineral salts, specifically formulated to make up for deficiencies or increased requirements not compensated by a balanced diet, is often inadequate, for example because of unpleasant organoleptic characteristics or low gastrointestinal tolerability. Said problems are particularly significant in the case of mineral salts such as iron, zinc and copper.

Iron, in particular, is a trace element essential to the body, and plays an important role in human nutrition.

An adult man's body contains about 3-4 g of iron, mainly (about 60%) present in the haemoglobin of the erythrocytes, for oxygen transport, and in ferritin, the protein involved in physiological iron storage. A minimal proportion of iron is also present in some enzymes as a prosthetic group, or as a cofactor necessary for the correct operation of enzymes.

In the event of reduced incorporation of iron in the erythrocytes, the body draws on its iron reserves to prevent the onset of the typical symptoms of anaemia. When the reserves are depleted or insufficient to meet the body's requirements, the ferritin and haemoglobin values fall, leading to an alteration in functional erythrocyte levels. In this situation of deficiency, even enzymes that need iron as a prosthetic group reduce their efficiency, to the extent of causing serious metabolic effects [Fuqua et al., 2012].

The absorption, transport and elimination processes are finely regulated at physiological level, giving rise to homeostasis of iron.

The dietary iron intake mainly falls into in two forms: haem iron, mainly present in foods of animal origin, and non-haem iron, which is present in cereals, pulses and green-leaved vegetables, and exists in the forms of divalent (ferrous) iron Fe³⁺ and trivalent (ferric) iron Fe³⁺. However, although plant sources contain a good store of iron, they do not represent the ideal source thereof in practice, due to the presence of substances such as oxalates and phytates which, when they bind to iron, prevent its absorption [Hurrel & Egli, 2010].

This is the main reason why those who follow a strict vegetarian or vegan diet are more likely to develop iron insufficiency or deficiency, even in the absence of underlying pathological conditions that affect iron absorption, transport and storage.

The process of absorption of dietary iron begins in the stomach, wherein the action of the gastric juices facilitates its dissociation from the food matrices with which it is complexed.

Iron is absorbed along the intestinal tract, especially in the duodenum. While iron can be absorbed directly from the enterocytes by endocytosis processes, the absorption of non-haem iron depends on its state of oxidation: it can only be absorbed if it is in the Fe³⁺ oxidation state; consequently, Fe³⁺ iron must be converted before being absorbed. Its conversion (or reduction) is known to take place mainly in the intestine, due to the action of duodenal cytochrome b (DCYTB), present on the apical domain of the duodenal cells; subsequently, a divalent metal transporter (DMT-1) enables it to enter the intestinal cell [EFSA Journal 2015; 13(10):4254]. The operation of DCYTB therefore limits the absorption of Fe³⁺, because cytochrome saturation can make the absorption of Fe³⁺ slower than that of divalent iron.

Iron elimination mainly takes place as a result of desquamation of the epithelium of the intestinal mucosa. The body therefore does not possess active mechanisms to eliminate excess iron; it absorbs the amount it requires, and eliminates it via the physiological turnover of the intestinal epithelium. It can thus be deduced that the aspect that influences the iron absorption process is its concentration in the body.

In conditions of insufficient dietary intake or an increased requirement (e.g. pathological conditions), one treatment option is intravenous or intramuscular iron administration; in this way the iron is readily bioavailable. However, said administration route is invasive and characterised by very poor compliance; it is therefore only used in cases of extreme and immediate necessity. Iron is more commonly administered orally.

Oral iron supplementation is the most convenient and commonly used way of supplementing the body's iron pool. It is performed by using products containing non-haem iron salts, namely divalent Fe³⁺ (e.g. iron sulphate, iron gluconate or iron fumarate) or trivalent Fe³⁺ (e.g. iron pyrophosphate). Divalent and trivalent iron salts are commercially available in pharmaceutical forms, especially swallowable capsules or tablets, and sachets for dissolving in water. Iron sulphate is one of the most widely used of the products on the market; it is the preferred form in pharmacological treatment because its bioavailability is greater than that of other salts.

Oral administration of iron sulphate, like other non-haem iron salts has limitations that reduce its efficacy.

A first limitation is a lower degree of absorption than that of haem iron. This limitation can be overcome or reduced by taking iron salts with meals, because the other substances present in food (e.g. vitamin C) promote its absorption.

A second limitation, which frequently accompanies the intake of iron salts, is the lower tolerability of the treatment. Individuals who use iron salts often complain of unpleasant symptoms, such as the perception of a metallic flavour in the mouth, and also of gastrointestinal side effects such as heaviness and gastric pain. This low tolerability is particularly evident in the more bioavailable iron salts, such as iron sulphate; stomac ache, nausea, vomiting, diarrhoea and abdominal pains are the most frequent adverse reactions recorded. Particularly sensitive individuals are advised not to use products based on iron sulphate, and it is recommended that it is taken with meals to limit the adverse effects. This leads to discontinuous treatment and/or, at worst, discontinuance of the treatment with the inevitable health consequences. In particular, it has been found that patient compliance with treatments based on iron tablets is fairly low in pregnant women due to its adverse gastrointestinal effects, partly due to the size of the tablets to be swallowed, which aggravates any nausea that may be present [Nguyen et al., 2008]. This limitation aggravates the deficiency, because an effective treatment based on iron salts requires a fairly long administration period, as it may take months to restore a suitable iron pool in the body.

In addition to said limitations, further aspects to be taken into account relate to the differences between divalent iron salts (Fe³⁺) and trivalent iron salts (Fe³⁺). Specifically, although divalent iron salts are generally fairly soluble and bioavailable, they have a very unpleasant metallic flavour and odour, and are also potentially unstable. Conversely, trivalent iron salts exhibit better tolerability and stability over time, but are characterised by lower solubility and bioavailability than divalent salts.

Techniques for preparing compositions containing mineral salts for oral use, including iron, have been proposed. Compositions containing iron salts which involve the use of various agents such as phospholipids (e.g. lecithin) or surfactants (e.g. sucrose esters) have been devised for the treatment of iron deficiency. WO 2014/009806 and WO 2018/189649 describe methods comprising numerous stages of preparation of iron-based compositions. EP 0 870 435 B1 discloses compositions obtained by methods consisting of numerous process stages, comprising the formation and purification of salts by means of neutralisation reactions. WO85/00664 describes a liposomal coating technology.

WO 2019171236 discloses formulations of lactoferrin and guanosine nucleotides wherein ferrous salts can optionally be present.

WO 2019171236 discloses enteric coatings mainly based on cellulose derivatives such as HPMC. A single example (example 21) describes a coating with shellac and alginic acid, without stating the specific properties thereof. Other examples describe mixtures of methacrylic copolymers and alginates.

The known techniques are mainly applied to prepare compositions in solution comprising trivalent iron salts. However, as stated above, the body can directly absorb non-haem divalent iron Fe³⁺, but not trivalent iron Fe³⁺, trivalent iron must therefore first be reduced to the divalent form in order to be absorbed. Said reduction reaction can take place at physiological level due to specific nutritional ingredients (e.g. vitamin C) which enable it to be converted. With the aid of vitamin C (ascorbic acid), trivalent iron is immediately reduced to divalent iron; for this reason, simultaneous consumption of suitable amounts of vitamin C is generally recommended to ensure that iron is absorbed effectively.

### DESCRIPTION OF THE INVENTION

The purpose of the present invention is to provide a solution that improves the organoleptic aspects and tolerability of mineral salts after oral administration, at the same time guaranteeing sufficient bioavailability, which is necessary to ensure that iron supplementation is effective for the user. The invention suggests the use of simple, convenient processes that do not alter the chemical nature of the ingredients and additives with health-giving properties. Surprisingly, the bioavailability of iron in the resulting granulate has proved similar to that of iron deriving from iron sulphate, the preferred form for pharmacological treatment due to its greater bioavailability.

The composition according to the invention comprises, in addition to said mineral salt, a soluble fibre with health-giving properties, preferably a soluble fructan.

Inulin is an example of a long-chain fructan with a degree of polymerisation of about 10, while fructooligosaccharides (FOS) are short-chain fructans generally having a degree of polymerisation ranging between 3 and 5. Fructooligosaccharides are generally used in products such as diet supplements due to their well-known health-giving properties. They are prebiotic fibres resistant to digestion in the gastrointestinal tract which arrive unmodified in the colon, where they are fermented by a limited number of bacteria, mainly bifidobacteria, promoting their growth and, by means of competition mechanisms, inhibiting the growth of pathogenic bacteria. It has been reported that prebiotic substances mitigate the adverse gastrointestinal effects of iron in children [Paganini, 2017]. They are usually available in diet supplements, and their dose ranges between 1 and 10 g/day.

The composition according to the invention comprises sodium alginate and shellac as well as mineral salt and soluble fructan.

Sodium alginate, extracted from seaweed cell walls, has the appearance of a gum. In the food industry it is classed as an additive and used as an emulsifying agent and thickener, but can be considered as a soluble fibre. Alginates are also used for their mucoprotective properties.

Shellac is a natural resin consisting of terpenes, obtained from the secretions of the insect Kerria lacca. The substance is soluble in basic aqueous solutions. As it is edible, shellac is widely used in the food industry as a polishing agent for pills and candies. It is classified as a food additive for said purpose, and is also used as a fruit coating to prevent deterioration after picking.

The weight percentage of soluble fructan in the composition according to the invention ranges between 20% and 70%, preferably between 35 and 55%, and most preferably between 40% and 45%.

The composition preferably comprises a soluble fructan selected from inulin and/or fructooligosaccharides. The fructan is advantageously in powder form, with a degree of polymerisation ranging between 3 and 10, more preferably between 3 and 5.

Sodium alginate is present in a weight percentage ranging between 1% and 10%, preferably between 1% and 5%, and more preferably between 1% and 2.5%.

Sodium alginate is present in an amount ranging between 1% and 10%, preferably between 1% and 5%, and more preferably between 1% and 2.5%.

Shellac is present in an amount ranging between 1% and 10%, preferably between 2% and 5%, and more preferably between 2% and 2.5%.

The sodium alginate to shellac ratio preferably ranges between 1:4 and 4:1.

According to a preferred aspect of the invention the mineral salt is iron, in order to make it tolerable for oral administration, in particular but not exclusively for sensitive individuals such as children, pregnant and lactating women, and individuals suffering from gastrointestinal disorders.

In an even more preferred aspect, the iron takes the form of divalent (ferrous) iron Fe²⁺.

The composition according to the invention comprises divalent iron salt Fe³⁺ in a percentage ranging between 25% and 75%, preferably between 40% and 60%, and most preferably between 50% and 55% by weight.

The divalent (ferrous) iron salt Fe²⁺ is preferably iron fumarate.

The composition according to the invention preferably comprises, or alternatively consists of, a divalent (ferrous) iron salt Fe²⁺, a soluble fructan, sodium alginate and shellac in the amounts stated above.

Most preferably, the composition according to the invention comprises, or alternatively consists of, iron fumarate, fructooligosaccharides, sodium alginate and shellac in the amounts stated above.

Advantageously, the compositions according to the invention take the form of a granule or micro-granule, made with fluid-bed granulation technology.

Said granulate or micro-granule can be suitably mixed with other substances and/or additives acceptable in the food and/or pharmaceutical industries, to advantageously provide an end product for oral use which takes the form of swallowable tablets, capsules, effervescent pharmaceutical forms, granulate designed to be reconstituted in water or dissolved directly in the mouth, softgels, syrups, solutions, suspensions or oral drops, packages in blister packs, pill boxes, bottles, sachets or stick packs, the various ingredients being selected in the appropriate physical form on the basis of the know-how of the skilled person.

A preferred aspect of the invention involves the production of chewable tablets containing said granulate. As illustrated above, patient compliance is a crucial aspect of iron deficiency treatment; in this respect, chewable tablets represent a more advantageous form than the widely available tablets and capsules, especially for children, individuals with swallowing difficulties, complicated in some cases by disorders (e.g. globus pharyngis), or individuals suffering from presbyphagia.

According to a further aspect thereof, the invention also provides a kit comprising:
a liquid phase acceptable in the food industry in a bottle, and
a powder phase in the measuring cap of said bottle, said cap containing all or part of a composition comprising a mineral salt, at least one soluble fructan, sodium alginate and shellac, and said bottle containing the remainder of said composition.

In a preferred embodiment, the kit according to the invention comprises a bottle including a measured amount of a liquid phase acceptable from the dietary standpoint and a measuring cap including a pre-set amount of the composition according to the invention.

The end product can be, for example, a food, a diet supplement, a special medical food, a medical device or a medicament.

It has been found that the compositions according to the invention possess organoleptic and iron tolerability characteristics, unexpectedly exhibiting bioavailability values similar to those of iron sulphate. In fact, as iron fumarate is notoriously far less water-soluble than iron sulphate (https://en.wikipedia.org/wiki/Iron(II)_fumarate; https://en.wikipedia.org/wiki/Iron(II)_sulfate) and has generically lower iron release than iron sulphate [Bannerman et al., 1996], it has surprisingly been observed that the majority of the iron released by the composition according to the invention is bioavailable in a similar percentage to iron sulphate.

The invention therefore improves the organoleptic characteristics of mineral salts, promoting their bioavailability.

### EXAMPLES

### Example I. Preparation of Granulate

A mixture of powders containing iron fumarate and FOS is prepared by accurately weighing the powders. The mixture is prepared in the rack of a fluid-bed granulator, and heated until an optimum temperature of 40-45° C. is reached. The mixture of powders is then introduced into the apparatus and kept in suspension by means of a continuous airstream; the granulation process subsequently takes place with water. At the end of said operation the resulting granules must be completely dried before the coating stage takes place. For this purpose, a solution obtained by dissolving precise amounts of sodium alginate and shellac in water is prepared separately; the solution is introduced into the spray nozzle of the apparatus and sprayed onto the moving powder mixture. The continuous airstream and regular spraying of the solution promote homogeneous distribution of the ingredients. Hot air subsequently causes the water to evaporate, and a granulate with the qualitative and quantitative composition shown in Table I is obtained at the end of the drying and cooling process

TABLE I

| Ingredient | Composition |
|---|---|
| Iron fumarate | 50% |
| Fructooligosaccharides (FOS) | 45% |
| Shellac Acquagold | 2.5% |
| Sodium alginate | 2.5% |

It was found that the compositions according to the invention possess excellent organoleptic and iron tolerability characteristics.

### Example II. Evaluation of Organoleptic Characteristics of the Granulate According to the Invention

A portion of the granulate described in Example I and a portion of commercially available iron fumarate, both equivalent to 14 mg of elemental iron (100% of Nutrient Reference Value—EU Reg. 1169/2011) were weighed separately using known procedures. The amounts are reported in Table II.

TABLE II

| Sample A | | Sample B | |
|---|---|---|---|
| | Amount (mg), | | Amount (mg), |
| | equivalent to 14 mg | | equivalent to 14 mg |
| Ingredient | of elemental iron | Ingredient | of elemental iron |
| Granulate | 96.6 mg | Iron fumarate | 45.1 mg |
| according to | | | |
| the invention | | | |

The following trial protocol was used to evaluate the organoleptic characteristics of the two samples compared.

General Criteria

Each of the compositions listed in Table II above was tested on a group of 4 subjects. The inclusion criteria for admission to the test were solely ethical: the subjects had to be over the age of majority, healthy, and not suffering from any taste and/or iron metabolism alterations correlated with disorders and/or illnesses.

Trial Procedure

Each subject independently tasted first one and then the other sample of the example, maintaining a suitable time interval (at least 2 hours) between one tasting and the next, and rinsing their mouth, so that the evaluation of each administration was not influenced by the previous one.

Evaluation Criteria

On each tasting, the following parameters were evaluated: metallic odour and flavour, gastrointestinal tolerability, aftertaste, and staining of teeth, tongue and/or palate. Each parameter was evaluated on a scale ranging from 0 (unsatisfactory) to 5 (highly satisfactory).

Iron odour
z1: unsatisfactory
2
3
4
5: highly satisfactory
Gastrointestinal tolerability
1 : intolerable
2
3
4
5: highly tolerable
Staining (e.g. teeth, tongue and/or palate)
1 : evident stains
2
3
4
5: no stains
Metallic flavour
1 : intolerable
2
3
4
5: imperceptible
Aftertaste
1: persistent aftertaste
2
3
4
5: no aftertaste

Results

The results are set out in Table III.

TABLE III

| Sample A | Sample B |
|---|---|
| Participant 1 | Participant 1 |
| Iron odour: 5 | Iron odour: 5 |
| Metallic flavour: 3 | Metallic flavour: 1 |
| Gastrointestinal tolerability: 5 | Gastrointestinal tolerability: 3 |
| Aftertaste: 4 | Aftertaste: 1 |
| Staining: 2 | Staining: 2 |
| Participant 2 | Participant 2 |
| Iron odour: 5 | Iron odour: 4 |
| Metallic flavour: 5 | Metallic flavour: 3 |
| Gastrointestinal tolerability: 5 | Gastrointestinal tolerability: 5 |
| Aftertaste: 5 | Aftertaste: 5 |
| Staining: 5 | Staining: 4 |
| Participant 3 | Participant 3 |
| Iron odour: 5 | Iron odour: 5 |
| Metallic flavour: 5 | Metallic flavour: 3 |
| Gastrointestinal tolerability: 5 | Gastrointestinal tolerability: 5 |
| Aftertaste: 4 | Aftertaste: 2 |
| Staining: 5 | Staining: 5 |
| Participant 4 | Participant 4 |
| Iron odour: 5 | Iron odour: 5 |
| Metallic flavour: 4 | Metallic flavour: 2 |
| Gastrointestinal tolerability: 5 | Gastrointestinal tolerability: 5 |
| Aftertaste: 4 | Aftertaste: 4 |
| Staining: 5 | Staining: 5 |

Examination of the data collected demonstrates that the composition according to the invention generally exhibited improved organoleptic characteristics compared with iron fumarate. In particular, a definite improvement as regards the perception of a metallic flavour was observed. 50% of participants reported an improvement in the metallic aftertaste.

Surprisingly, it was found that the compositions containing iron fumarate exhibit similar bioavailability to that of iron sulphate.

### Example III. Release and Bioavailability Study

The release profile and bioavailability of the composition according to the invention were analysed and measured by comparison with iron sulphate (the preferred form for pharmacological treatment) using an in vitro system, under conditions simulating the gastrointestinal environment.

The tests were conducted according to methods published in the international scientific literature and internally validated.

For the measurement of the comparative release profile, amounts of the two test samples were first weighed, both equivalent to 30 mg of elemental iron. The samples were initially placed in contact with a solution of HCl (0.1N) and incubated at a temperature of 37±0.5° C. A solution of Na₂HPO₄ was then added to the samples to buffer the pH to a value of 6.8. Said conditions were maintained throughout the remainder of the experiment. To evaluate the release profile of the iron, the two samples were centrifuged at 10,000 rpm at pre-set time intervals such as 1, 2, 4, 6 and 24 h. At the end of centrifugation the samples were taken up and analysed to evaluate the iron release profiles. The iron determination analyses were conducted with the ICP-OES THERMO FISHER ICAP 6300. Table IV shows the results of the release test.

TABLE IV

| | IRON RELEASED | |
|---|---|---|
| TIME (h) | Ferrous sulphate | Granulate according to the invention |
| 1 | 84.16 ± 1.07 | 73.99 ± 0.87 |
| 2 | 85.22 ± 0.98 | 77.96 ± 1.10 |
| 4 | 100.03 ± 0.20 | 79.87 ± 0.94 |
| 6 | — | 80.46 ± 1.57 |
| 24 | — | 80.53 ± 1.44 |

The in vitro bioavailability study was conducted by the dialysis membrane method. Said method, by its nature, is based on the physicochemical characteristics of the environments wherein the desired substance passes from one compartment to the other, and takes no account of any active mechanisms or biological interaction. The method involves three successive simulated digestion stages: buccal digestion in the presence of amylase, gastric digestion in the presence of pepsin, and finally, intestinal digestion in the presence of pancreatin.

Buccal Digestion

In order to simulate buccal digestion, suitable amounts of the test samples were placed in contact with 10 mg of amylase and 1.5 mL of PBS at pH 6.9 (10-3 M). The resulting mixtures were inserted into dialysis membranes (Spectrum Laboratories Inc., USA, MWCO: 12-14,000 Daltons), tightly closed at each end and immersed in vials containing PBS at pH 6.9. The samples were then incubated at a temperature of 37±0.5° C. for 5 minutes.

Gastric Digestion

After incubation, HCl (0.85N), pepsin and a solution of NaN₃ (0.04% w/w) were added to the open membranes. The membranes were closed, placed in vials containing HCl (0.85N), and incubated at 37±0.5° C. for 2 h.

Intestinal Digestion

After said 2 h period, a solution of NaHCO₃ (0.8 M) and pancreatin was added. The closed membranes were placed in vials containing PBS at pH 7.0, and incubated at a temperature of 37±0.5° C. for a further 4 h.

To evaluate the in vitro bioavailability of the iron, the solution contained in the vials was taken up after each digestion step, and the samples were analysed with an ICP-OES THERMO FISHER ICAP 6300.

Bioavailability is defined as the percentage of iron recovered in the bioaccessible fraction after in vitro digestion, in relation to the original undigested sample, and calculated with the equation: (bioaccessible fraction/total content)×100%. Table V shows the results of the bioavailability test.

TABLE V

BIOACCESSIBLE FRACTIONS

| | | Granulate according |
|---|---|---|
| DIGESTION | Ferrous sulphate | to the invention |
| Buccal | 8.61 ± 0.73 | 0.06 ± 0.03 |
| Gastric | 65.81 ± 1.39 | 71.36 ± 1.12 |
| Intestinal | 7.16 ± 0.41 | 7.64 ± 1.11 |
| Bioavailability (%) | 81.6% ± 2.53 | 79.1% ± 2.26 |

The results demonstrated that, despite the different release profile, the cumulative bioavailability values of the three digestion stages (buccal, gastric and intestinal) were similar for the two samples tested. This demonstrates that the granulate according to the invention does not modify the release profile and bioavailability of iron compared with ferrous sulphate.

### Example IV. Chewable Tablets

The active ingredients and excipients, in powder form, are precisely weighed and mixed in a mechanical mixer in the composition reported in Table VI. The resulting uniform mixture flows by gravity from a hopper to be introduced into the tablet press cavity.

TABLE VI

| Ingredient | Composition | |
|---|---|---|
| Granulate according to the invention | 206.40000 | mg |
| Vitamin C | 147.70000 | mg |
| Copper gluconate | 11.20000 | mg |
| Folic acid | 0.55236 | mg |
| Sorbitol | 1,294.14764 | mg |
| Sucralose | 1.00000 | mg |
| Silicon dioxide | 1.00000 | mg |
| Orange flavouring | 30.00000 | mg |
| Magnesium stearate | 28.00000 | mg |
| Maltodextrins | 280.00000 | mg |

Tablet weight: 2.00000 g
Tablet format: round, diameter 18
Colour: white with brick red dots

### Example V. Orodispersible Stick Packs

Active ingredients and excipients commonly used in the food industry, in powder form, are precisely weighed and mixed in a mechanical mixer in the composition reported in Table VII. The resulting uniform pre-measured mixture flows by gravity from a hopper to be introduced into the stick pack wrapping.

TABLE VII

| Ingredient | Composition | |
|---|---|---|
| Granulate according to the invention | 206.40000 | mg |
| Vitamin C | 100.00000 | mg |
| Vitamin B12 | 2.10000 | mg |
| Folic acid | 0.43400 | mg |

| | |
|---|---|
| Xylitol | q.s. |
| Sucralose | q.s. |
| Orange flavouring | q.s. |
| Silicon dioxide | q.s. |
| Maltodextrins | q.s. |

Tablet weight: 1.50000 g
Colour: white with brick red dots

### Example VI. Assessment of the Organoleptic Characteristics of the Stick Packs Containing the Granulate According to the Invention

The following trial protocol was used to evaluate the organoleptic characteristics of the granulate according to the invention, inserted in a sample of orodispersible product as described in Example V.

General Criteria

The product described in Example V was tested on a group of 7 subjects. The inclusion criteria for admission to the test were solely ethical; the subjects had to be over the age of majority, healthy, and not affected by taste and/or iron metabolism alterations correlated with disorders and/or illnesses.

Trial Procedure

Each person independently tasted the product of Example V, after a suitable time had elapsed since the last intake of food or drinks, in order to evaluate each parameter sequentially, leaving a suitable interval (at least 2 hours) to elapse so that some parameters, such as gastric tolerability and aftertaste, could be evaluated.

Evaluation Criteria

On each tasting, the following parameters were evaluated: metallic odour and flavour, gastrointestinal tolerability, aftertaste, and staining of teeth, tongue and/or palate. Each parameter was evaluated on a scale ranging from 0 (unsatisfactory) to 5 (highly satisfactory).

Iron odour
1: unsatisfactory
2
3
4
5: highly satisfactory
Gastrointestinal tolerability
1 : intolerable
2
3
4
5: highly tolerable
Staining (e.g. teeth, tongue and/or palate)
1: evident stains
2
3
4
5: no stains
Metallic flavour
1 : intolerable
2
3
4
5: imperceptible
Aftertaste
1: persistent aftertaste
2
3
4
5: no aftertaste

Results

The results are set out in Table VIII.

TABLE VIII

Participant 1
Iron odour: 5
Metallic flavour: 5
Gastrointestinal tolerability: 4
Aftertaste: 4
Staining: 5
Participant 2
Iron odour: 5
Metallic flavour: 4
Gastrointestinal tolerability: 4
Aftertaste: 4
Staining: 5
Participant 3
Iron odour: 4
Metallic flavour: 5
Gastrointestinal tolerability: 5
Aftertaste: 4
Staining: 5
Participant 4
Iron odour: 5
Metallic flavour: 5
Gastrointestinal tolerability: 4
Aftertaste: 4
Staining: 5
Participant 5
Iron odour: 5
Metallic flavour: 5
Gastrointestinal tolerability: 5
Aftertaste: 4
Staining: 5
Participant 6
Iron odour: 5
Metallic flavour: 3
Gastrointestinal tolerability: 5
Aftertaste: 4
Staining: 5
Participant 7
Iron odour: 5
Metallic flavour: 4
Gastrointestinal tolerability: 5
Aftertaste: 5
Staining: 5

The data collected demonstrate that the composition according to the invention was advantageously inserted in a formulation of the product, confirming the optimum organoleptic characteristics of the granulate according to the invention, already found in Example II; in particular, excellent tolerability was found in terms of metallic odour, flavour and aftertaste.

### BIBLIOGRAPHY

Bannerman Judy, Campbell Norman R. C., Hasinoff Brian B., Venkataram Suresh. The dissolution of iron from various commercial preparations. Pharmaceutica Acta Helvetiae 1996 July; Vol. 71, Issue 2, Pages 129-133.
EFSA Journal 2015; 13(10):4254. Scientific Opinion on Dietary Reference Values for iron. EFSA Panel on Dietetic Products, Nutrition and Allergies (NDA). European Food Safety Authority (EFSA), Parma, Italy.
Fuqua Brie K, D Vulpe Christopher, J Anderson Gregory. Intestinal iron absorption. J Trace Elem Med Biol. 2012 June; 26(2-3):115-9.
Hurrell Richard, Egli Ines. Iron bioavailability and dietary reference values. Am J Clin Nutr. 2010 May; 91(5):1461S-1467S.
Nguyen Patricia, Nava-Ocampo Alejandro, Levy Amalia, O'Connor Deborah L, Einarson Tom R, Taddio Anna, Koren Gideon. Effect of iron content on the tolerability of prenatal multivitamins in pregnancy. BMC Pregnancy Child birth. 2008 May 15; 8:17.
Paganini Daniela, Zimmermann Michael B. The effect of iron fortification and supplementation on the gut microbiome and diarrhea in infants and children: a review. AM J Clin Nutr. 2017 December; 106(Suppl 6):1688S-1693S.

1. Composition in the form of a granulate or micro-granule comprising a mineral salt selected from iron, zinc and copper salt and soluble fibers selected from soluble fructans having a degree of polymerization ranging between 3 and 10, said granulate or micro-granule being coated with shellac and sodium alginate.

2. Composition according to claim 1 wherein the mineral salt is an iron salt.

3. Composition according to claim 13 wherein the divalent iron salt is iron fumarate.

4. (canceled)

5. Composition according to claim 1, wherein said soluble fructan is selected from inulin and/or fructooligosaccharides.

6. Composition according to claim 5, wherein the weight percentage of fructans to the total composition ranges from 20% to 70%.

7. Composition according to claim 1, wherein the weight percentage of sodium alginate to the total composition ranges from 1% to 10%, the weight percentage of shellac to the total composition ranges from 1% to 10%, and the weight ratio of sodium alginate to shellac ranges between 1:4 and 4:1.

8. Composition according to claim 1, wherein the weight percentage of mineral salt to the total composition ranges from 25% to 75%.

9. A kit comprising:
a. a liquid phase, acceptable in the food industry, in a bottle, and
b. a powder phase in the measuring cap of said bottle, said cap containing a composition according to claim 1.

10. A food diet supplement, medical food or medical device comprising the compositions according to claim 1.

11. The food diet supplement, medical food or medical device according to claim 10 in the form of swallowable tablets, chewable tablets, capsules, effervescent pharmaceutical forms, granules to be reconstituted in water or to be dissolved in the mouth, softgels, syrups, solutions, suspensions or oral drops.

12. The composition according to claim 1, wherein said soluble fructans have a degree of polymerization between 3 and 5.

13. The composition according to claim 1, wherein the mineral salt is a divalent iron salt.
